SEQUENCE LISTING (7 sequences)

SEQ ID NO: 1 - DNA, 106 bp, Artificial Sequence
  Target nucleic acid
agggcattttggacaaagcgtctacgctgcagtcctcgctcactgggcacggtgagcgtgaacacaaaccccaaaatccccttagtcagaggtgacaggattggtc

SEQ ID NO: 2 - DNA, 24 bp, Artificial Sequence
  Reverse primer
agggcattytggacaaakcgtcta

SEQ ID NO: 3 - DNA, 22 bp, Artificial Sequence
  Forward primer
gaccratcctgtcacctctgac

SEQ ID NO: 4 - DNA, 24 bp, Artificial Sequence
  Probe
caccgtgcccagtgagcgaggact

SEQ ID NO: 5 - DNA, 70 bp, Artificial Sequence
  Target nucleic acid
cctggcacccagcacaatgaagatcaagatcattgctcctcctgagcgcaagtactccgtgtggatcggc

SEQ ID NO: 6 - DNA, 18 bp, Artificial Sequence
  Forward primer
cctggcacccagcacaat

SEQ ID NO: 7 - DNA, 20 bp, Artificial Sequence
  Reverse primer
gccgatccacacggagtact

(19) United States
(12) Patent Application Publication
(10) Pub. No.: US 2020/0239944 A1
(43) Pub. Date: Jul. 30, 2020

(54) PARTICLE WITH UCST MATERIAL APPLIED THERETO, AND NUCLEIC ACID AMPLIFICATION METHOD USING SAME

(71) Applicant: KOREA INSTITUTE OF SCIENCE AND TECHNOLOGY, Seoul (KR)

(72) Inventors: Sang Kyung KIM, Seoul (KR); Junsun KIM, Seoul (KR)

(73) Assignee: KOREA INSTITUTE OF SCIENCE AND TECHNOLOGY, Seoul (KR)

(21) Appl. No.: 16/628,346

(22) PCT Filed: Jul. 3, 2018
(86) PCT No.: PCT/KR2018/007523

§ 371 Date: Apr. 8, 2020

(30) Foreign Application Priority Data
Jul. 4, 2017 (KR) 10-2017-0084595
Jul. 2, 2018 (KR) 10-2018-0076483

(51) Int. Cl. C12Q 1/6837; C12Q 1/686; C12Q 1/6853

(52) U.S. Cl. CPC C12Q 1/6837; C12Q 1/6853; C12Q 1/686

(57) ABSTRACT

In the present invention, when amplifying a nucleic acid by incorporating at least one primer among a forward primer and reverse primer and/or a probe in an upper critical solution temperature (UCST) particle, or when amplifying a nucleic acid by incorporating at least one primer among the forward primer and reverse primer and/or a probe in a UCST particle and fixing to a hydrogel fine particle, same the primer or probe comprised in the UCST particle can be discharged within a certain temperature range. Accordingly, the formation of primer dimers can be prevented while also achieving excellent PCR amplification efficiency.

### CROSS-REFERENCE TO RELATED APPLICATION

This application claims priority to Korean Patent Application No. 10-2017-0084595 filed on Jul. 4, 2017 and Korean Patent Application No. 10-2018-0076483, filed on Jul. 2, 2018, and all the benefits accruing therefrom under 35 U.S.C. §119, the contents of which in its entirety are herein incorporated by reference.

### TECHNICAL FIELD

Disclosed is related to a UCST particle to which is introduced a novel nucleic acid supplying material and which comprises a primer, a hydrogel fine particle with the primer fixed by comprising the UCST particle, a nucleic acid amplification method using the same, and a nucleic acid amplification apparatus using the same.

### BACKGROUND ART

Techniques for simultaneously analyzing many target nucleic acids in one channel by using polymerase chain reaction (PCR) have been developed. Solid-phase nucleic acid amplification technology (Solid-Phase PCR) is a technique for simultaneously analyzing several target nucleic acids, and a primer or a probe is used by being fixed to the solid surface. In the case of the solid-phase nucleic acid amplification technology, compared with a case where primers in one direction are fixed to the surface of a solid, and primers in the other direction or probes are supplied to a solution, if forward and reverse primers or probes are all fixed to the surface of the solid, there is a problem in that efficiency of amplification reaction very poor. Meanwhile, in a case where primers in one direction or probes are fixed to the surface of the solid, and primers in the other direction or probes are introduced into a solution, there is a problem in that the primers form primer dimers which are nonspecific binding to each other and interfere with amplification signals.

### [Description of National Support Research and Development]

This research was conducted by the support of the National Research Council of Science & Technology (Research operating expense support of the National Research Council of Science & Technology (Ministry of Science, ICT and Future Planning), the development of a national disaster-type livestock disease field diagnosis technology, task specific number: 1711046486) under the supervision of the Korea Institute of Science and Technology.

### CITATION LIST

### [Patent Literature]

Korean Patent Publication No. 10-2015-0048964 (May 11, 2015)

### SUMMARY OF INVENTION

### [Technical Problem]

An object of the present invention is to provide a UCST particle and a hydrogel fine particle that prevent formation of a primer dimer and have excellent amplification efficiency of a target nucleic acid.

### [Solution to Problem]

In order to achieve the above object, an aspect of the present invention provides an upper critical solution temperature (UCST) particle for PCR, comprising: a UCST polymer matrix having an upper critical solution temperature (UCST) at 20° C. to 90° C.; and at least one primer among a forward primer and a reverse primer of target nucleic acid as a polymerase chain reaction (PCR) primer or a probe of target nucleic acid, dispersed in the UCST polymer matrix.

An aspect of the present invention provides a hydrogel fine particle for PCR to which at least one primer among a forward primer and a reverse primer of target nucleic acid as a polymerase chain reaction (PCR) primer or a probe of target nucleic acid is fixed, in which the hydrogel fine particle comprises the UCST particle, which is fixed to the fine particle, at least one primer among the forward primer and the reverse primer or the probe of target nucleic acid is comprised in the UCST particle and is fixed to the hydrogel fine particle, and the hydrogel fine particle has a porous structure that comprises pores.

An aspect of the present invention provides a nucleic acid amplification apparatus comprising at least one UCST particle.

An aspect of the present invention provides a nucleic acid amplification apparatus comprising at least one hydrogel fine particle.

An aspect of the present invention provides a method of preparing the UCST particle, comprising a step of introducing at least one primer among a forward primer and a reverse primer of target nucleic acid or a probe of target nucleic acid to an upper critical solution temperature (UCST) material in a liquid phase, and performing mixing and coagulation to prepare the UCST particle.

An aspect of the present invention provides a method of preparing the hydrogel fine particle, comprising: a step of introducing at least one primer among a forward primer and a reverse primer of target nucleic acid or a probe of target nucleic acid into an upper critical solution temperature (UCST) material in a liquid phase and performing mixing and coagulation to prepare the UCST particle; a pre-polymer solution preparation step of mixing the prepared UCST particle, a hydrogel monomer, and a photo initiator to prepare a pre-polymer solution, in which the solution further comprises a primer that is not comprised in the UCST particle among the forward primer and the reverse primer when only one primer among the forward primer and the reverse primer is comprised in the UCST particle; and a step of ejecting the pre-polymer solution in a droplet form and curing the solution to prepare the hydrogel fine particle.

An aspect of the present invention provides a nucleic acid amplification method comprising: a step of injecting the UCST particle into a reaction chamber; a step of injecting a solution comprising at least one target nucleic acid into the reaction chamber; and a step of subjecting the target nucleic acid to a polymerase chain reaction (PCR) to amplify the target nucleic acid.

An aspect of the present invention provides a nucleic acid amplification method comprising: a step of injecting at least one hydrogel fine particle into a reaction chamber; a step of injecting a solution comprising at least one target nucleic acid into the reaction chamber; and a step of subjecting the target nucleic acid to a polymerase chain reaction to amplify the target nucleic acid.

### [Advantageous Effects of Invention]

The UCST particles according to the present invention comprise at least one primer among forward and reverse primers or a probe, and thus can stably store all primers and/or probes in the UCST particles, before a denaturation step of PCR, particularly a one-step reverse transcription PCR process, so it is possible to prevent the formation of a primer dimer caused by nonspecific binding occurring between a primer and a primer or between a primer and a probe. The hydrogel fine particles comprising the UCST particles of the present invention can prevent the formation of a primer dimer by fixing all of the forward and reverse primers and/or probes to the hydrogel fine particles and supplying primers and/or probes required for the amplification reaction of target nucleic acid only to the corresponding particles. Also, if the primer comprised in the UCST particles in the denaturation step of a PCR process is released out into the hydrogel fine particles, the primer has a high degree of freedom and can exhibit high amplification efficiency in the nucleic acid amplification, as in a case where only primers in one direction are fixed to the hydrogel fine particle and primers in the other direction are introduced into a solution.

### BRIEF DESCRIPTION OF DRAWINGS

FIG. 1 is a diagram schematically illustrating the principle of performing one-step reverse transcription-quantitative PCR (One-step RT-qPCR) by using UCST particles comprising reverse primers as an embodiment of the present invention.

FIG. 2 is a diagram illustrating a UCST particle comprising a reverse primer and a probe as an embodiment of the present invention.

FIG. 3 is a diagram schematically illustrating the principle in which a hydrogel fine particle comprises a UCST particle therein, the UCST particle is melted at the temperature of the UCST or higher during PCR, and the primer comprised in the UCST particle leaks to perform the nucleic acid amplification reaction, as an embodiment of the present invention.

FIGS. 4A and 4B are graphs illustrating fluorescence signals as results of performing one-step reverse transcription-quantitative PCR, immediately after the preparation of UCST particles, which are one embodiment of the present invention, and after one day, two days, and six days from the preparation. The y axis of FIG. 4A represents absolute values of each fluorescence signal immediately after the preparation of UCST particles, after one day, two days, and six days from the preparation, the y axis of FIG. 4B represents standardized values of each fluorescence intensity immediately after the preparation of UCST particles, after one day, two days, and six days from the preparation, and the x axes of FIGS. 4A and 4B represent cycle numbers.

FIG. 5 is a graph (red line, dark line) illustrating results of performing one-step reverse transcription-quantitative PCR using UCST particles comprising reverse primers as an embodiment of the present invention, and results of performing one-step reverse transcription-quantitative PCR in which a reaction solution comprising both reverse primers and forward primers without UCST particles is introduced into a channel is used as Comparative Example 1 (blue line, light line). In FIG. 5, solid lines represent positive control signals, dotted lines represent, as false positive control signals generated by the primer dimers, no template control signals generated without the reaction of a template, the x axis represents a cycle number, and the y axis represents a standardized fluorescence intensity.

FIG. 6 is a graph illustrating results of performing one-step reverse transcription-quantitative PCR using UCST particles comprising a reverse primer and a probe as an embodiment of the present invention. In the graph of FIG. 6, the x axis represents a cycle number, and the y axis represents an absolute value of the fluorescence signal.

FIG. 7 is a diagram illustrating an appearance when a UCST material is melted and fluorescent labeled nucleic acid comprised therein is released out of the hydrogel fine particles, when the temperature of hydrogel fine particles comprising UCST particles is increased to the UCST or higher, as an embodiment of the present invention.

FIG. 8 is a diagram for analyzing a degree of decrease of fluorescence when a UCST material is melted and fluorescent labeled nucleic acid comprised therein is released out of hydrogel fine particles when the temperature of hydrogel fine particles comprising UCST particles is increased to the UCST or higher, as an embodiment of the present invention.

FIG. 9 is a diagram for analyzing a nucleic acid amplification reaction of hydrogel fine particles comprising agarose as a UCST material, as an embodiment of the present invention.

FIG. 10 is a diagram for analyzing a nucleic acid amplification reaction of hydrogel fine particles comprising gelatin as a UCST material, as an embodiment of the present invention.

FIG. 11 is a diagram for analyzing a nucleic acid amplification reaction of hydrogel fine particles comprising LMPA as a UCST material as an embodiment of the present invention.

FIG. 12 is a diagram for analyzing a nucleic acid amplification reaction of hydrogel fine particles comprising PEG-aCD as a UCST material as an embodiment of the present invention.

FIG. 13 is a diagram illustrating a result of comparing PCR efficiencies of an embodiment of the present invention (one primer immobilized with UCST) and Comparative Examples 2 and 3 (pair primer immobilized, and one primer immobilized without UCST).

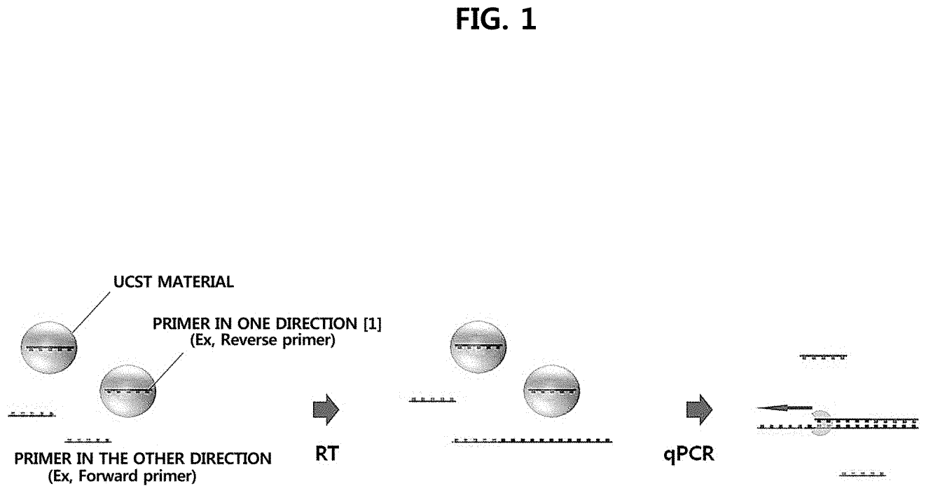

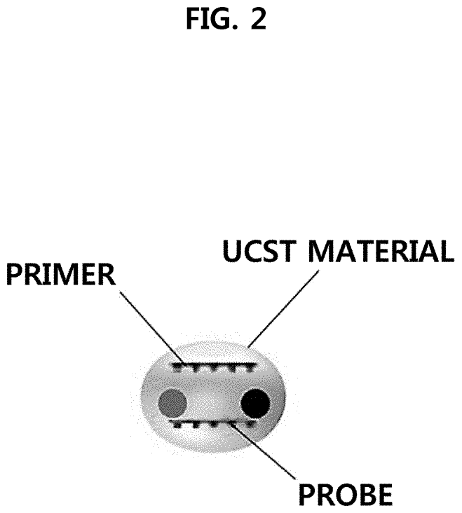

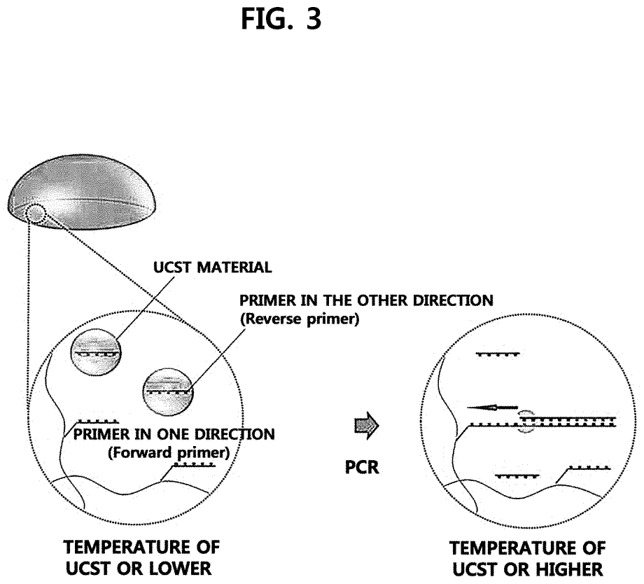

### DESCRIPTION OF EMBODIMENTS

Hereinafter, in order to enable a person having ordinary skill in the art of the present invention to easily carry out the present invention, preferred embodiments of the present invention are described in detail.

An embodiment of the present invention may be a UCST particle for PCR comprising: a UCST polymer matrix having a upper critical solution temperature (UCST) at 20° C. to 90° C.; and at least one primer among forward primers and reverse primers of target nucleic acid as a polymerase chain reaction (PCR) primer or a probe of the target nucleic acid, dispersed in the UCST polymer matrix. The UCST particles may further comprise reverse transcription (RT) primers in the UCST particles, or a forward primer or a reverse primer, which is the PCR primer, may be used as the reverse transcription primer without further comprising the reverse transcription primer.

One embodiment of the present invention may be a hydrogel fine particle for PCR to which at least one primer among a forward primer and a reverse primer of target nucleic acid as a polymerase chain reaction (PCR) primer or a probe of target nucleic acid is fixed, in which the upper critical solution temperature (UCST) particles is comprised and fixed, and the at least one primer among the forward and reverse primers or the probe of the target nucleic acid may be comprised in the UCST particles and fixed to the hydrogel fine particles. In addition, the hydrogel fine particles may comprise two or more UCST particles, and each of the two or more UCST particles may comprise at least one among the forward primer, the reverse primer, and the probe of the target nucleic acid.

In the present specification, the “upper critical solution temperature (UCST) particle” refers to a particle made of a material having temperature sensitivity, which is made of a material present in a solid phase at a temperature of a critical solution temperature or lower and in a liquid phase above the critical solution temperature. In addition, the UCST particles may be particles having the upper critical solution temperature (UCST) at 20° C. to 90° C. and may be particles phase-transitioned from a solid phase to a liquid phase or a liquid phase to a solid phase at the upper critical solution temperature. In one embodiment, the UCST particles may be particles made of a material that is melted in a denaturation step of a PCR process.

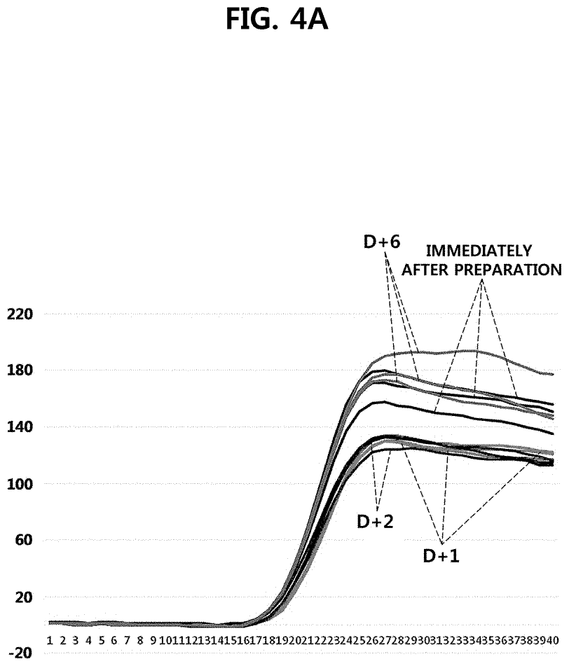

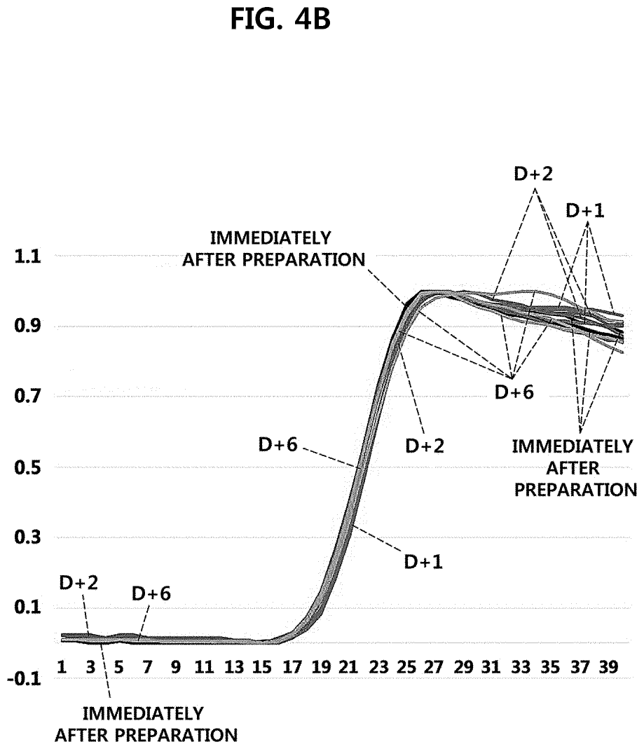

In an embodiment of the present invention, the UCST particles are prepared to comprise a primer or a probe, and it was confirmed that the primer or the probe can be stably stored in the UCST particle until 6 days have elapsed after the preparation (Experimental Example 1, FIGS. 4A and 4B).

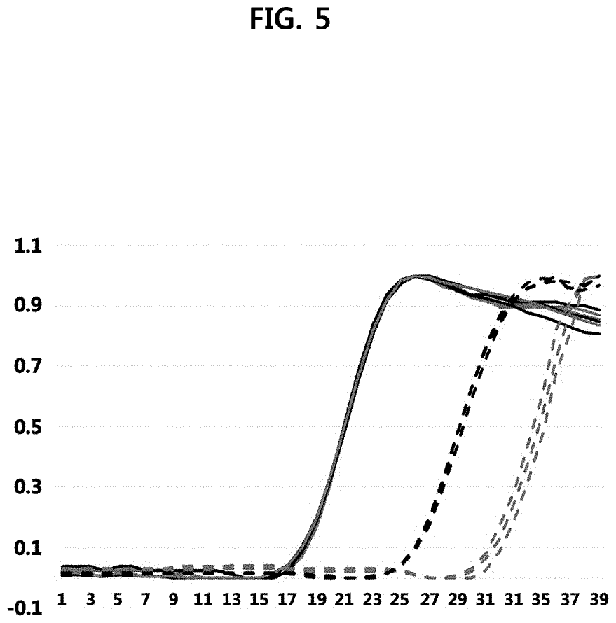

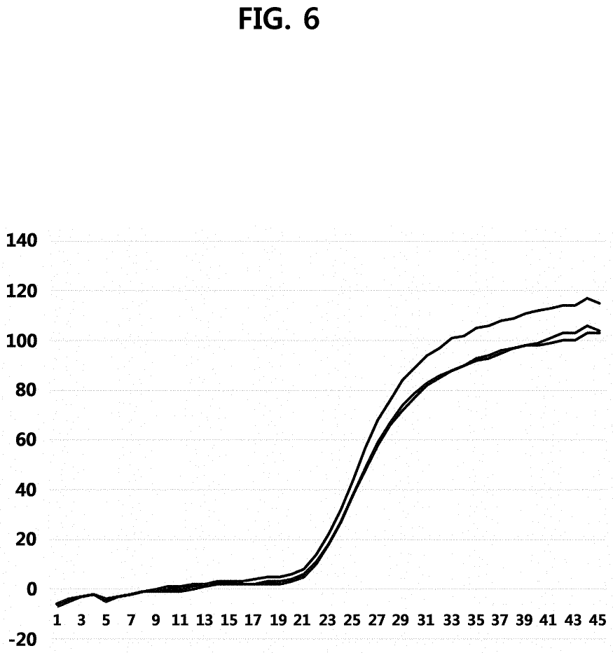

In addition, in one-step reverse transcription-quantitative PCR (One-step RT-qPCR), it is known that a reverse transcription reaction and quantitative PCR are sequentially performed in one reaction vessel, primers which are used in both of the reverse transcription and the quantitative PCR reaction and primers which are used only in the quantitative PCR are mixed in one reaction vessel from the start, and thus the primers to be participated only in the quantitative PCR reaction nonspecifically act to probes which are used in the reverse transcription reaction, or primers, target nucleic acid (RNA), or the like which are used in the reverse transcription reaction, to occur problems. Therefore, according to an embodiment of the present invention, in the course of PCR by using UCST particles comprising reverse primers or UCST particles comprising reverse primers and probes, in the temperature of a threshold temperature or lower, that is, before the denaturation step of the PCR process, the primer or the probe comprised in the UCST particles is not released out of the UCST particles, and thus the UCST particles can very stably comprise the primers or the probes immediately before the PCR reaction. Therefore, it is confirmed that a false positive signal caused by the primer dimer in the course of PCR is suppressed, and the nucleic acid amplification reaction, particularly, the one-step reverse transcription-quantitative PCR can be more sensitively and accurately performed (Experimental Examples 2 and 3, and FIGS. 5 and 6).

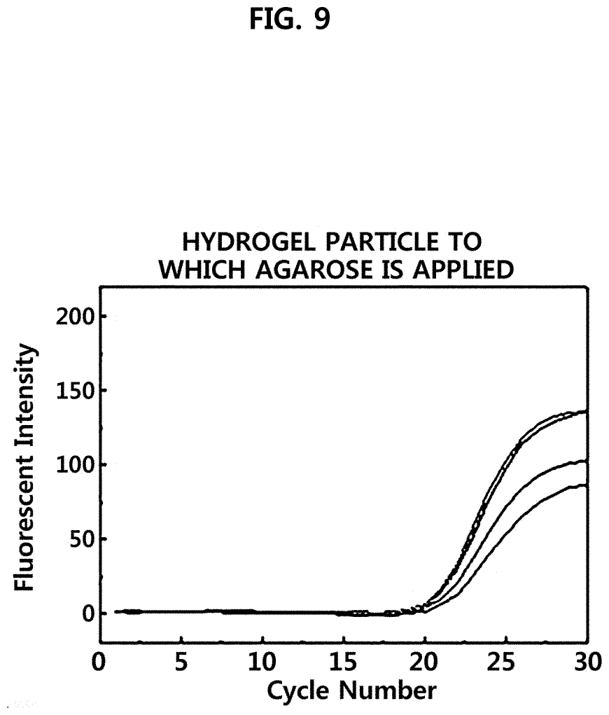

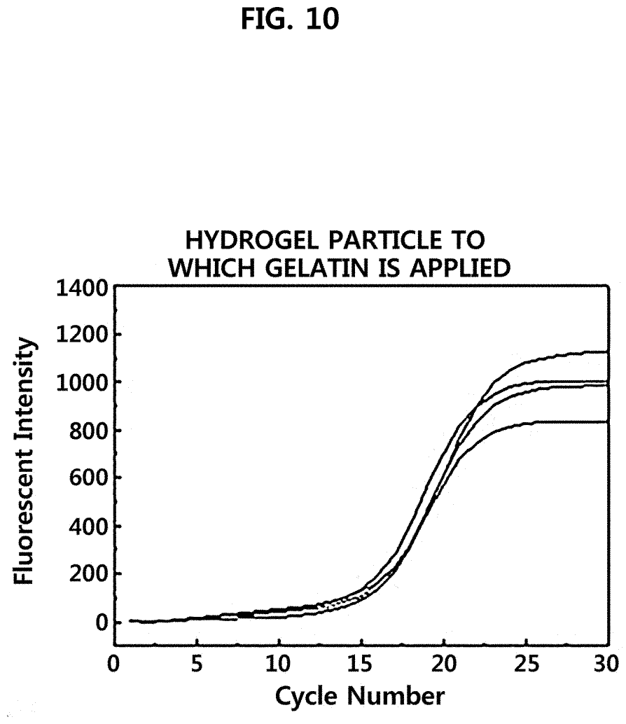

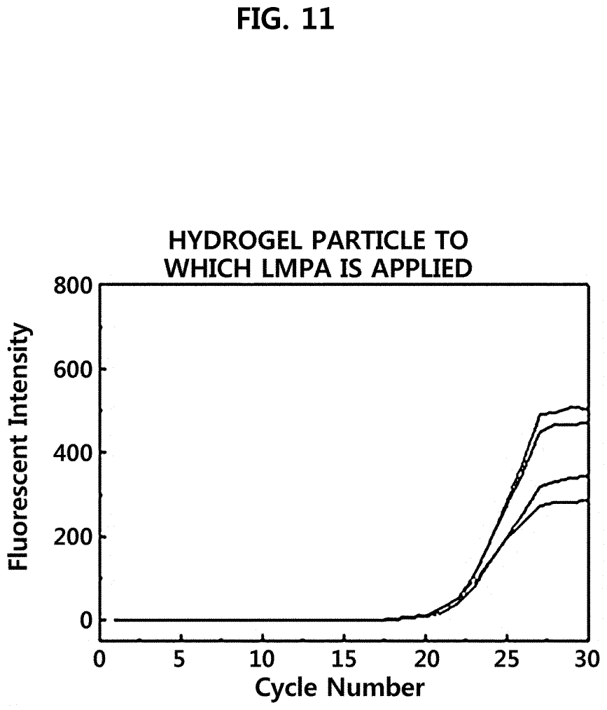

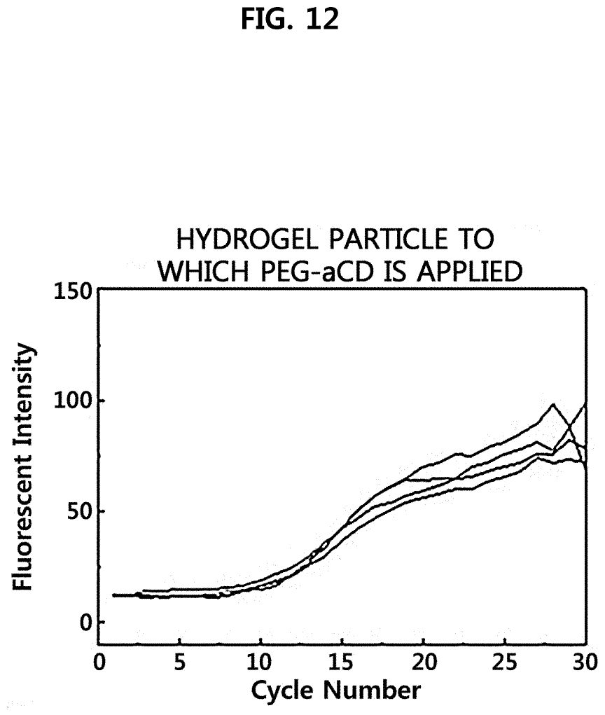

Additionally, according to an embodiment of the present invention, as at least one primer among the forward primers and the reverse primers or probes are comprised inside the UCST particles and fixed to the hydrogel fine particles, in the temperature of the critical solution temperature or lower, that is, before the denaturation step of the PCR process, the primers or the probes comprised in the UCST particles are not released out of the UCST particles and the UCST particles can very stably comprise the primers or the probes immediately before the nucleic acid amplification reaction. Thus, nonspecific signals generated by different target nucleic acid primers or probes, and primers can be effectively prevented. In the temperature of the critical solution temperature or higher, that is, in the denaturation step of the PCR process, the UCST particles are melted, and the primers or probes comprised inside the UCST particles are released into the hydrogel fine particle pores. Therefore, it can freely move of the primers or the probes inside or outside of the hydrogel fine particles, and thus the nucleic acid amplification reaction with high efficiency can be performed (Experimental Examples 4 to 6, and FIGS. 9 to 13).

For example, the UCST particle may comprise one or more selected from the group consisting of agarose, gelatin, collagen, low melting point agarose (LMPA), and PEG-aCD (a mixture of polyethylene glycol and alpha-cyclodextrin). The material for forming the UCST particle is not limited to the materials exemplified above, and various UCST materials can be selected according to room temperature stability and nucleic acid amplification reactivity. Specifically, the UCST particle may comprise agarose having a critical solution temperature of 80° C. to 90° C., gelatin having a critical solution temperature of 40° C. to 50° C., collagen having a critical solution temperature of 30° C. to 40° C., and the like, as an embodiment of a natural material. In another embodiment, the UCST particle may comprise LMPA having a critical solution temperature of 60° C. to 80° C., as a material obtained by modifying a natural material. In another embodiment, the UCST particle may comprise PEG-aCD having a critical solution temperature of 20° C. to 90° C. and the like, as a material that generates UCST properties due to molecular attraction.

According to an embodiment of the present invention, the hydrogel fine particle may have a porous structure comprising pores. If the hydrogel fine particle is a porous structure comprising pores, the porosity of the fine particle as an embodiment may be 10 volume % to 80 volume %, more specifically 20 volume % to 70 volume % with respect to the total volume of the fine particles. If the porosity is out of the range, the porosity is poor or the structural stability of the fine particles is unstable, such that the nucleic acid amplification reaction may be disadvantageous.

According to an embodiment, examples of the target nucleic acid primer comprise at least one nucleic acid of DNA, RNA, LNA, and PNA, but the present invention is not limited thereto. In this case, the target nucleic acid primer may be 10 to 100 base pairs (bp), more specifically 20 to 50 base pair, but the sequence type and the sequence length of the nucleic acid primers may be modified without limitation depending on the target nucleic acid.

According to an embodiment of the present invention, the hydrogel fine particle may comprise one primer of the forward primer and the reverse primer inside the UCST particle, and in this case, the other primer which is not comprised inside the UCST particle may be fixed to the outside of the hydrogel fine particle or to the inside of the pore of the hydrogel fine particle. According to an embodiment of the present invention, the hydrogel fine particle may comprise the probe of the target nucleic acid inside the UCST particle. In this case, one primer of the forward primer and the reverse primer is fixed to the inside of the pore of the hydrogel fine particle or both of the forward primer and the reverse primer are not fixed to the inside of the pore of the hydrogel fine particle. Specifically, the primer that is not comprised inside the UCST particle may be fixed to the outside of the hydrogel fine particle or to the inside of the pore by comprising a hydrogel fine particle fixing functional group at the 5′ end or a 3′ end of the primer sequence or by being cross-linked with the hydrogel. The primer may be fixed by a covalent bond or a peptide bond to a hydrogel monomer inside the pore of the hydrogel fine particle. According to an embodiment, the functional group that can be linked by a covalent bond to the hydrogel monomer may comprise acrydite. According to another embodiment, a functional group that may be linked by a peptide bond to the hydrogel may comprise an amine group, a carboxyl group, or the like. According to an embodiment, a primer that modifies an acrydite functional group at a 5′ end of the sequence may be fixed to the hydrogel when the hydrogel monomer is converted to a polymer in the preparation of the hydrogel fine particle.

According to an embodiment of the present invention, a particle diameter of the hydrogel fine particle may be, for example, 10 μm to 500 μm on average, more specifically 100 μm to 300 μm on average. The shape is not limited as long as it is a three-dimensional structure, and specifically, examples thereof comprise a spherical shape, a hemispherical shape, a disc shape, and a plate shape. The material of the hydrogel fine particle can be used without limitation as long as the material is a polymer (pre-polymer) that can be solidified. Specifically, hydrophilic polymers such as polyethylene glycol-diacrylate (PEG-DA) or polyacrylamide (PA) can be used.

As a PCR fluorescent labeling method, a nonspecific fluorescent labeling method in which SYBR green I dyes or the like are used and caused to intercalate between amplification products generated during the nucleic acid amplification reaction to exhibit fluorescence, and a specific fluorescent labeling method in which a probe is used and bound to a specific sequence of an amplification product generated by the nucleic acid amplification reaction and exhibits fluorescence by the nucleic acid amplification reaction may be used.

If the specific fluorescent labeling method is used as an embodiment of the present invention, the UCST particle may comprise a probe inside thereof. Specifically, the probe may be a selective fluorescent probe. The fluorescent probe is bound to target nucleic acid, provides a fluorescence signal, and detects target nucleic acid in real time. According to an embodiment, as the target nucleic acid is amplified by the polymerase chain reaction, the fluorescence intensity also increases, and thus, target nucleic acid amplified can be quantified by detecting the fluorescence intensity. The fluorescent probe can be used without limitation as long as the fluorescent probe is complementarily bound to target nucleic acid and exhibits fluorescence. For example, the selective fluorescent probe may comprise TaqMan probe or the like. According to an embodiment, the TaqMan probe is nucleic acid in which an 5′ end is modified with a fluorescence material (FAM and the like), and a 3′ end is modified with a quencher material (BHQ and the like), and is specifically hybridized to a template DNA in an annealing step, but fluorescence generation is suppressed by a quencher on the probe. The TaqMan probe hybridized to the template is degraded due to 5′-3′ exonucleolytic activity provided by a Taq DNA polymerase during an extension reaction, the fluorescent dye is freed from the probe, the suppression by the quencher is released, and thus fluorescence is exhibited.

According to an embodiment of the present invention, the UCST particle or the hydrogel fine particle may further comprise one or more of an encoder providing information of a target nucleic acid primer or a probe and a fluorescent marker providing quantitative information of amplified nucleic acid in the UCST particle or in the pore of the fine particle. The encoder refers to a material that distinguishes the nucleic acid primer and the probe in each of the UCST particle or the hydrogel fine particle by colors or shapes. For example, dyes or quantum dots that exhibit various colors of fluorescence, and metal, plastics, glass, and silicon which have a specific shape may be used. Otherwise, without using the encoder, the target nucleic acid primers or the probes in each of the UCST particle or the hydrogel fine particle can be distinguished by changing the size or the shape of the UCST particle or the hydrogel fine particle or providing specific marking on the surface of the UCST particle or the hydrogel fine particle. Alternatively, the target nucleic acid primer or the probe in each of the UCST particle or the hydrogel fine particle can be distinguished by specifying a position of the UCST particle or each hydrogel fine particle in an array.

According to an embodiment of the present invention, the method of preparing the UCST particle may comprise a step of introducing at least one primer among a forward primer and a reverse primer of target nucleic acid or a probe of target nucleic acid into a UCST material in a liquid phase, and performing mixing and coagulation to prepare a UCST particle. The step of preparing the UCST particle may further comprise a step of heating the UCST material to a critical solution temperature or higher and completely dissolving the UCST material to a liquid phase before at least one among the forward primer and the reverse primer of the target nucleic acid or the probe is introduced into and mixed with the UCST material in the liquid phase. According to an embodiment, the coagulating the UCST material after the mixing may comprise a step of cooling the UCST material to a critical solution temperature or lower and coagulating the UCST material into a solid phase.

According to an embodiment of the present invention, the method of preparing the hydrogel fine particle may comprise a step of introducing at least one primer among the forward primer and the reverse primer or the probe of the target nucleic acid into the UCST material in the liquid phase, and performing mixing and coagulation to prepare the UCST particle; a step of mixing the prepared UCST particle, the hydrogel monomer, and a photo initiator to prepare a pre-polymer solution; and a step of ejecting the pre-polymer solution in a droplet form and curing the solution to prepare a hydrogel fine particle. In this case, when only one primer of the forward primer and the reverse primer is comprised in the UCST particle, in the step of preparing the pre-polymer solution, the pre-polymer solution may further comprise a primer of the forward primer and the reverse primer which is not comprised in the UCST particle. In addition, when neither of the forward primer and the reverse primer is comprised in the UCST particle or only the probe of the target nucleic acid is comprised in the UCST particle, the pre-polymer solution may further comprise one primer of the forward primer and the reverse primer.

In the present specification, the “pre-polymer” means a preliminary polymer in which the polymerization or polycondensation reaction is stopped in an appropriate step for an easier molding process of the polymer. In the case of the present invention, the pre-polymer means a polymer before solidification in a state in which a molding process is easy.

According to an embodiment, the method may further comprise a washing step after the hydrogel fine particle is prepared, and the UCST material that is not coagulated and a porogen can be removed by the washing step.

According to an embodiment, the step of ejecting the pre-polymer solution in a droplet form may comprise a method using a microchannel, a piezo method, a solenoid valve method, microspotting, or the like, and accordingly, fine particles in various forms and sizes can be prepared.

According to an embodiment of the present invention, if different kinds of target nucleic acid primers or probes are injected depending on the kind of the target nucleic acid by using the method of preparing the UCST particle, a plurality of UCST particles comprising different target nucleic acid primers or probes can be prepared. The UCST particle may further comprise one or more of the encoder providing information of the target nucleic acid primer and the probe comprised in each UCST particle and the fluorescent marker providing quantitative information of the amplified target nucleic acid.

According to an embodiment of the present invention, if different kinds of the target nucleic acid primer or the probe are injected depending on the kinds of the target nucleic acid by using the method of preparing the hydrogel fine particle, a plurality of hydrogel fine particles comprising different target nucleic acid primers or probes can be prepared. In addition, the pre-polymer solution may further comprise one or more of an encoder providing information of a target nucleic acid primer or a probe comprised in each hydrogel fine particle and a fluorescent marker providing quantitative information of amplified target nucleic acid.

According to an embodiment, the step of preparing the pre-polymer solution may further comprise a porogen in the solution. According to an embodiment, the step of preparing the pre-polymer solution may further comprise changing the size of the porogen comprised in the solution to adjust the size of the pore formed in the hydrogel fine particle. In this case, polyethylene glycol (PEG) and polyacrylamide (PAM) may be used as the porogen. As the polyethylene glycol, specifically, PEG200, PEG300, PEG400, PEG600, PEG1000, PEG1500, PEG2000, PEG3000, PEG3350, PEG4000, PEG6000, PEG8000, PEG10000, PEG12000, PEG20000, PEG35000, PEG40000, and the like (manufactured by Sigma Aldrich Corporation) may be used.

According to an embodiment, the curing of the hydrogel fine particle is curing while maintaining the shape of the hydrogel fine particle before curing. As long as the shape can be maintained, the method may be optical, chemical or thermal curing and is not limited thereto, and examples thereof comprise curing by ultraviolet.

According to an embodiment of the present invention, it is possible to provide a nucleic acid amplification apparatus that comprises at least one UCST particle. According to an embodiment, the nucleic acid amplification apparatus may comprise a plurality of UCST particles each comprising a primer or a probe for different target nucleic acids.

According to an embodiment of the present invention, it is possible to provide a nucleic acid amplification apparatus comprising at least one hydrogel fine particle. According to an embodiment, the nucleic acid amplification apparatus may comprise a plurality of hydrogel fine particles each comprising a primer or a probe for different target nucleic acids.

According to an embodiment, the apparatus may further comprise a reaction chamber, and the reaction chamber may comprise an array or a tube in which the UCST particles or the hydrogel fine particles are arranged. The material of an array according to an embodiment may be glass, plastic, a polymer, silicon, or the like, and the kind thereof is not limited as long as the temperature condition of the nucleic acid amplification reaction can be applied.

According to an embodiment of the present invention, it is possible to provide a nucleic acid amplification method comprising a step of injecting at least one UCST particle to a reaction chamber; a step of injecting a solution comprising at least one target nucleic acid to the reaction chamber; and a step of subjecting the target nucleic acid to a polymerase chain reaction (PCR) to amplify the target nucleic acid.

According to an embodiment, the step of amplifying the target nucleic acid may comprise melting the UCST particle in the denaturation step of the PCR and releasing a primer or a probe comprised inside thereof into the inside of the chamber. According to an embodiment, the step of amplifying the target nucleic acid may comprise melting the UCST particle in the denaturation step of the PCR and releasing the primer or the probe comprised inside thereof out of the UCST particle.

According to an embodiment of the present invention, it is possible to provide a nucleic acid amplification method comprising a step of injecting at least one hydrogel fine particle into a reaction chamber; a step of injecting a solution comprising at least one target nucleic acid into the chamber; and a step of subjecting the target nucleic acid to the polymerase chain reaction (PCR) and amplifying the target nucleic acid.

According to an embodiment, the step of amplifying the target nucleic acid may comprise melting the UCST particle in the denaturation step of the PCR and releasing the primer or the probe comprised inside thereof to the inside of the hydrogel fine particle pore.

According to an embodiment, the at least one UCST particle may each comprise at least one of a forward primer, a reverse primer, and a probe for a same target nucleic acid or may each comprise primers or probes for different target nucleic acids. According to an embodiment, the hydrogel fine particle may comprise two or more UCST particles, and the two or more UCST particles may each comprise at least one of a forward primer, a reverse primer, and a probe for a same target nucleic acid. According to an embodiment, the at least one hydrogel fine particle may each comprise primers or probes for different target nucleic acids. According to an embodiment, the solution comprising the target nucleic acid may further comprise a primer comprising locked nucleic acid (LNA) such as a 3′-locked nucleic acid primer, Tag polymerase, or the like. According to an embodiment, the reaction chamber may comprise an array or a tube in which the UCST particles or the hydrogel fine particles are arranged.

According to an embodiment, the method may further comprise a step of analyzing nucleic acid polymerized in each of the at least one UCST particle. According to an embodiment, the method may further comprise a step of analyzing nucleic acid polymerized in each of the at least one hydrogel fine particle. According to another embodiment, the method may further comprise a step of performing quantitative analysis in real time of the nucleic acid polymerized in each of one or more of the UCST particles or the hydrogel fine particles polymerized simultaneously with the polymerase chain reaction step, and different kinds of target nucleic acids may be amplified, and at the same time, detected and quantitatively analyzed in real time.

Hereinafter, the present invention is specifically described with reference to preparation examples, examples, and experimental examples. These preparation examples, examples, and experimental examples are provided only for exemplifying the present invention, and it is obvious to those skilled in the art, that the scope of the present invention are not construed to be limited by these preparation examples, examples, and experimental examples.

### PREPARATION EXAMPLE 1

### Preparation of UCST Particle Comprising Primer in One Direction

A UCST particle (Example 1) comprising a reverse primer was prepared by using LMPA as a UCST material, as an embodiment of the present invention.

Specifically, after the LMPA was completely melted to a liquid phase at a critical solution temperature or higher, and a reverse primer solution of target nucleic acid was introduced into the UCST material in the liquid phase by 10% v/v with respect to the total volume of the UCST material in the liquid phase and cooled down to the critical solution temperature or lower to be coagulated to prepare the UCST particle in the solid phase.

Sequences of the target nucleic acid and the reverse primer used at this time were as follows.

-Target nucleic acid:
(SEQ NO. 1)
5′-AGGGCATTTTGGACAAAGCGTCTACGCTGCAGTCCTCGCTCACTGGG
CACGGTGAGCGTGAACACAAACCCCAAAATCCCCTTAGTCAGAGGTGACA
GGATTGGTC-3′
-Reverse primer:
(SEQ NO. 2)
5′-AGGGCATTYTGGACAAAKCGTCTA-3′

### PREPARATION EXAMPLE 2

### Preparation of UCST Particle Comprising Primer in One Direction and Probe

A UCST particle (Example 2) comprising a reverse primer and a probe was prepared by using LMPA as a UCST material, as an embodiment of the present invention.

Specifically, a UCST particle in a solid phase comprising the reverse primer of SEQ NO. 2 and a probe of SEQ NO. 4 for the target nucleic acid of SEQ NO. 1 was prepared in the same method as in Preparation Example 1.

-Probe:
(SEQ NO. 4)
5′-CACCGTGCCCAGTGAGCGAGGACT-3′

### PREPARATION EXAMPLE 3

### Preparation of Hydrogel Fine Particle Comprising UCST Particle

Hydrogel fine particles comprising a UCST particle of Example 3-6 were prepared by using four kinds of UCST materials of agarose, gelatin, LMPA, and PEG-aCD, respectively, as an embodiment of the present invention.

Sequences of the target nucleic acid, the forward primer, and the reverse primer were as follows, and TaqMan™ probe was used as the probe. Acrydite was used as the forward primer and fixed to inside of the hydrogel fine particle.

-Target nucleic acid:
(SEQ NO. 5)
5′-CCTGGCACCCAGCACAATGAAGATCAAGATCATTGCTCCTCCTGAGC
GCAAGTACTCCGTGTGGATCGGC-3′
-Forward primer:
(SEQ NO. 6)
(5′Acryd)-CCTGGCACCCAGCACAAT-3′
-Reverse primer:
(SEQ NO. 7)
5′-GCCGATCCACACGGAGTACT-3′

Specifically, the UCST materials were completely melted to a liquid phase at each threshold melting temperature or higher, the reverse primer solution was introduced into the UCST materials in the liquid phase by 10% v/v with respect to the total volume of the UCST materials in the liquid phase, and cooled down to each threshold melting temperature or lower and coagulated to prepare UCST particles in the solid phase.

20% v/v of the UCST particles in the solid phase which comprises the reverse primer, 5% v/v of the forward primer solution, 40% v/v of poly(ethylene glycol) (PEG, Sigma-Aldrich Corporation, MW 600) as porogen, 20% v/v of poly(ethylene glycol)diacrylate (PEG-DA, Sigma-Aldrich Corporation, MW 700) as a hydrogel monomer, 5% v/v of 2-hydroxy-2-methyl propiophenone (Sigma-Aldrich Corporation) as a photo initiator, and 10% v/v of a buffer (PBS, etc.) with respect to the total volume of the hydrogel solution were mixed to prepare 100 μL of a pre-polymer hydrogel solution in total.

The prepared solution was ejected in the droplet form and was exposed to UV (360 nm wavelength, 35 mJ/cm²) for one minute to be cured to prepare hydrogel fine particles having average particle diameter of 400 μm. The fine particles were washed with PBS 1X buffer to remove uncured materials.

### EXPERIMENTAL EXAMPLE 1

### Confirmation of Storage Stability of Primer or Probe of UCST Particles

In order to confirm that the UCST particle according to an embodiment of the present invention has storage stability of the primer or the probe, the one-step reverse transcription-quantitative PCR (One-step RT-qPCR) was performed as follows.

The UCST particle of Example 1 prepared in [Preparation Example 1] was injected into a channel filled with PBS 1X buffer, and stored in the condition with the temperature of 40° C. applied. The corresponding UCST particles and the following forward primer of SEC NO. 3 were injected to the channel to perform the one-step reverse transcription-quantitative PCR immediately after the preparation, and after one day, two days, and six days from the preparation.

Forward primer:
(SEQ NO. 3)
5′-GACCRATCCTGTCACCTCTGAC-3′

The one-step reverse transcription-quantitative PCR was performed in the conditions of 42° C. and 10 minutes for a reverse transcription process, 95° C. and 4 seconds for a denaturation step of the quantitative PCR process, and 55° C. and 30 seconds for a DNA synthesis step (extension) of the quantitative PCR process. The denaturation step and the DNA synthesis step of the quantitative PCR process were repeated 40 times. The fluorescence intensity of the particles was observed 40 times in total for one second immediately after the DNA synthesis step of the quantitative PCR process, the fluorescence intensity was measured, and the results thereof are illustrated in FIGS. 4A and 4B.

As illustrated in FIGS. 4A and 4B, as a result of analyzing the same target nucleic acid (RNA), it was found that, from immediately after the preparation till after six days, the fluorescence intensity data were exactly identical, and the intensity of the fluorescence signals was also maintained. If the primer comprised in the UCST particle is released outside, the fluorescence intensity may be decreased or a Ct value may be delayed, but the fluorescence intensity was maintained and a Ct value was not delayed. This means that the primer was stably stored in the UCST particle of the present invention.

### EXPERIMENTAL EXAMPLE 2

### Nucleic Acid Amplification Reaction Using UCST Particles Comprising Primer

According to an embodiment of the present invention, the one-step reverse transcription-quantitative PCR (one-step RT-qPCR) was performed as the nucleic acid amplification reaction in the same method as in Experimental Example 1 by using the UCST particles comprising the reverse primers.

However, the UCST particles used was the UCST particles of Example 1 prepared in [Preparation Example 1]. At this time, as Comparative Example 1, without the UCST particles, the reaction solution comprising the reverse primer and the forward primer was introduced into the channel, and the one-step reverse transcription-quantitative PCR was performed in the same process as in Experimental Example 1.

Results of performing the one-step reverse transcription-quantitative PCR of Example 1 and Comparative Example 1 are illustrated in FIG. 5. Red lines (dark lines) of FIG. 5 indicate results of the one-step reverse transcription-quantitative PCR of Comparative Example 1, blue lines (light lines) indicate results of the one-step reverse transcription-quantitative PCR using the UCST particles of the present invention, solid lines indicate positive control signals, and dotted lines indicate no template control signals generated without reaction of templates which are false positive control signals by the primer dimer.

As illustrated in FIG. 5, since solid lines of Comparative Example 1 and Example 1 are exactly matched with each other, it is confirmed that, even if the one-step reverse transcription-quantitative PCR is performed by using the UCST particle comprising a primer in one direction, for example, a reverse primer, the same positive signal as in the existing one-step reverse transcription-quantitative PCR in the liquid phase is exhibited.

It is known that, in the one-step reverse transcription-quantitative PCR, the reverse transcription reaction and the quantitative PCR are sequentially performed in one reaction vessel, the primer (forward primer in Experimental Example 2) used in both of the reverse transcription and the quantitative PCR reaction and the primer (reverse primer in Experimental Example 2) used only in the quantitative PCR are mixed in one reaction vessel from the start, and thus the primers to be participated only in the quantitative PCR reaction nonspecifically act to the forward primer or target nucleic acid (RNA) during the reverse transcription reaction to cause problems.

However, when comparing the dotted lines of Comparative Example 1 and Example 1 of FIG. 5, Ct values of Example 1 are delayed by 6 or more, and this delay indicates the effect of increasing the sensitivity by about 100 times or more, so it is understood that, when the one-step reverse transcription-quantitative PCR is performed by using the UCST particles of the present invention, the false positive signals caused by the primer dimer are suppressed, and the nucleic acid amplification reaction can be performed more sensitively and accurately.

### EXPERIMENTAL EXAMPLE 3

### Nucleic Acid Amplification Reaction Using UCST Particles Comprising Primer and Probe

According to an embodiment of the present invention, the one-step reverse transcription-quantitative PCR (one-step RT-qPCR) as the nucleic acid amplification reaction was performed as follows using the UCST particles comprising the reverse primer and the probe.

Specifically, the one-step reverse transcription-quantitative PCR is performed in the same method as in Experimental Example 1, except that the UCST particles of Example 2 that comprises a reverse primer and a probe were used instead of the UCST particles of Example 1.

Results of performing the one-step reverse transcription-quantitative PCR of Example 2 are illustrated in FIG. 6.

As illustrated in FIG. 6, since results of three times of independent experiments in the environment with target RNA are exactly matched, it is confirmed that the one-step reverse transcription-quantitative PCR can be performed by using UCST particles comprising a primer and a probe.

### EXPERIMENTAL EXAMPLE 4

### Confirmation on Whether Primer was Released Depending on Temperature of UCST Particles in Hydrogel Fine Particles

In order to confirm whether the UCST particle according to an embodiment of the present invention releases the primer comprised inside the UCST particle in a specific temperature condition, the following experiment was performed.

After the hydrogel fine particles of Example 4 which is prepared in [Preparation Example 3] was injected into the channel filled with PBS 1X buffer, the fluorescence of the particles was observed for 100 seconds while the temperature was applied in the condition of 80° C. The decrease of the fluorescence of the particle means that the primer comprised in the UCST particles are released out of the hydrogel fine particle.

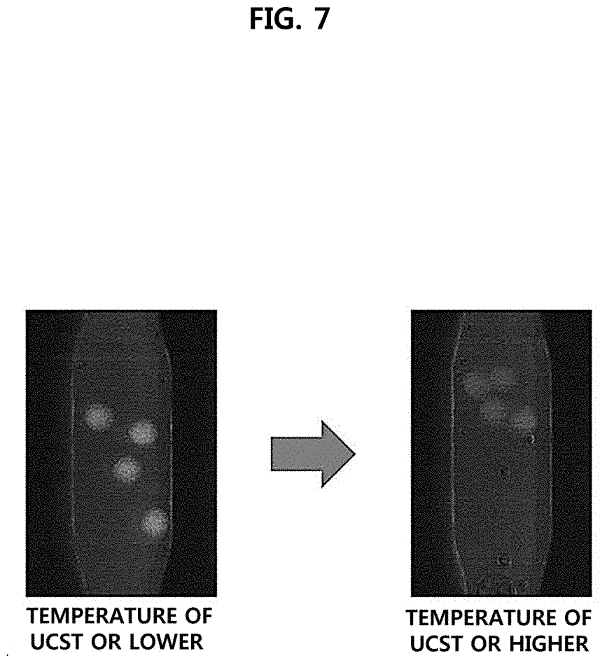

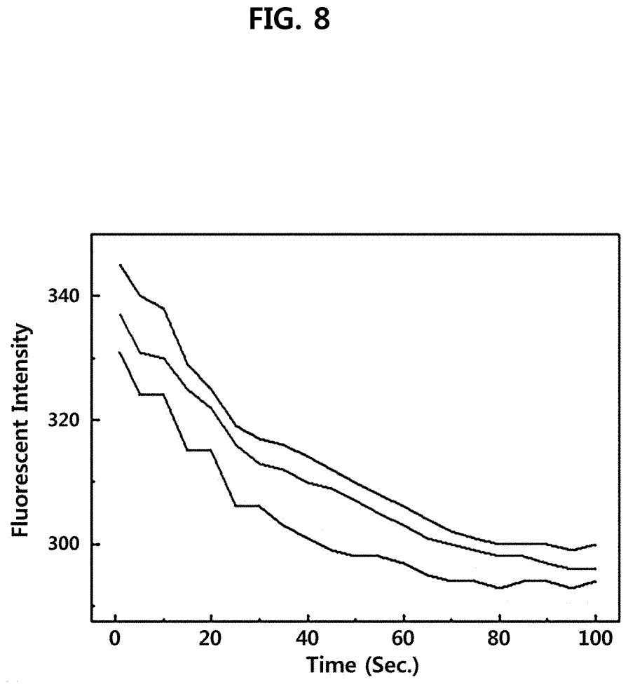

As a result, as illustrated in FIGS. 7 and 8, the fluorescence intensity decreased according to the elapse of time. FIG. 8 illustrates results of repeating the same experiment independently for three times by using the same fine particles. The results mean that according to the elapse of time, the UCST is melted around the UCST temperature, that is, at 40° C. to 50° C. which is the temperature condition of the denaturation step of the PCR process, such that the primer comprised in the UCST particle is released and freely participate in the amplification reaction.

### EXPERIMENTAL EXAMPLE 5

### Nucleic Acid Amplification Reaction Using Hydrogel Fine Particle Comprising UCST Particle

According to an embodiment of the present invention, the nucleic acid amplification reaction was performed by using the hydrogel fine particle comprising various UCST particles.

Each hydrogel fine particle of Examples 3 to 6 prepared in [Preparation Example 3] was introduced to a channel, a sample (nucleic acid template) to be analyzed was mixed with a PCR master mix (reagent comprising enzyme, buffer, dNTP, Mg2+, and the like) and introduced into the channel, and then PCR was performed.

At this time, the PCR was performed for 30 cycles of denaturation at 95° C. for 4 seconds, annealing and elongation at 60° C. for 30 seconds.

As a result, as illustrated in FIGS. 9 to 12, it was confirmed that the nucleic acid amplification reaction was effectively performed with all of Examples 3 to 6. Four graphs of FIGS. 9 to 12 illustrate results obtained by independently repeating the same experiments for four times. Even though the experiments were independent to each other, a large portion of graphs overlap each other, indicating it is possible to obtain constant and stable reaction results.

### EXPERIMENTAL EXAMPLE 6

### Comparison Of Amplification Efficiency When Nucleic Acid Amplification Reaction is Performed by Using Hydrogel Fine Particles Comprising UCST Particles

According to an embodiment of the present invention, the hydrogel fine particles (one primer immobilized with UCST) of Example 4 prepared in [Preparation Example 3] were used.

As the comparative example, hydrogel particles (pair primer immobilized; Comparative Example 2) which were the same as Example 4 except that the UCST particles were not comprised and both of the forward and reverse primers were fixed to the inside of the hydrogel fine particle pores, and hydrogel particles (one primer immobilized without UCST; Comparative Example 3) which were the same as Example 4 except that forward primers were fixed to the inside of the hydrogel fine particle pores without using UCST particles and do not comprise a reverse primer and a probe were comprised were used.

PCR was performed with each hydrogel particles of Example 4 and Comparative Examples 2 and 3 by the method provided in Experimental Example 5. At this time, concentrations of nucleic acid templates injected into each of Example 4 and Comparative Examples 2 and 3 were the same.

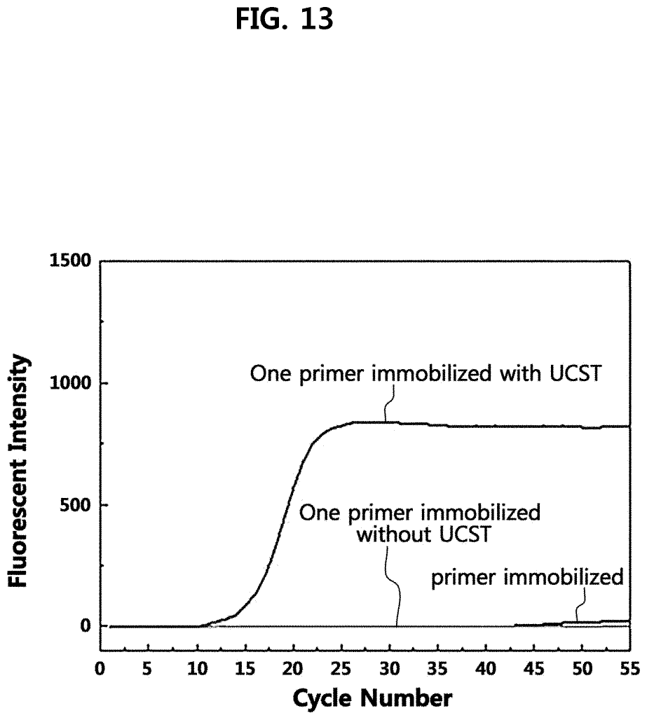

FIG. 13 illustrates results of the above PCR, and it is understood that, in the cases of Comparative Examples 2 and 3 in which UCST particles were not used, regardless of whether the primers in one direction were fixed to the inside of the hydrogel fine particles, very low fluorescence intensity was exhibited to show that the nucleic acid amplification reaction was hardly performed. Meanwhile an embodiment of the present invention exhibits very high PCR efficiency. It is considered that, this is because, in the present invention, by using the UCST particle, the formation of a primer dimer caused by a nonspecific bond generated between primers or between a primer and a probe is prevented, so the primer comprised in the UCST particle has a high degree of freedom during the PCR.

1. An upper critical solution temperature (UCST) particle for PCR, comprising:
a UCST polymer matrix having an upper critical solution temperature (UCST) at 20° C. to 90° C.; and
at least one primer among a forward primer and a reverse primer of target nucleic acid as a polymerase chain reaction (PCR) primer or a probe of target nucleic acid, dispersed in the UCST polymer matrix.

2. The UCST particle according to claim 1, comprising:
one or more selected from the group consisting of agarose, gelatin, collagen, low melting point agarose (LMPA), and a mixture of polyethylene glycol and alpha-cyclodextrin (PEG-aCD).

3. The UCST particle according to claim 1,
wherein the UCST particle is melted in a denaturation step of a PCR process.

4. The UCST particle according to claim 1,
wherein the probe is a selective fluorescent probe.

5. A hydrogel fine particle for PCR to which, at least one primer among a forward primer and a reverse primer of target nucleic acid as a polymerase chain reaction (PCR) primer, or a probe of target nucleic acid is fixed,
wherein the hydrogel fine particle comprises the upper critical solution temperature (UCST) particle according to claim 1, which is fixed to the fine particle,
at least one primer among the forward primer and the reverse primer or the probe is comprised in the UCST particle and is fixed to the hydrogel fine particle, and
the hydrogel fine particle has a porous structure that comprises pores.

6. The hydrogel fine particle according to claim 5,
wherein one primer of the forward primer and the reverse primer is comprised in the UCST particle, and
the other primer that is not comprised in the UCST particle is fixed to an inside of the pore of the hydrogel fine particle.

7. The hydrogel fine particle according to claim 6,
wherein the primer that is not comprised in the UCST particle is fixed by a covalent bond or a peptide bond to a hydrogel monomer inside the pore of the hydrogel fine particle.

8. A nucleic acid amplification apparatus comprising:
at least one UCST particle according to claim 1, and
a reaction chamber in which the UCST particle is arranged.

9. The nucleic acid amplification apparatus according to claim 8, comprising:
a plurality of UCST particles each comprising a primer or a probe for different target nucleic acids.

10. A nucleic acid amplification apparatus comprising:
the hydrogel fine particle according to claim 5; and
a reaction chamber in which the hydrogel fine particle is arranged.

11. A method of preparing the UCST particle according to claim 1, comprising:
a step of introducing at least one primer among a forward primer and a reverse primer of target nucleic acid or a probe of target nucleic acid to an upper critical solution temperature (UCST) material in a liquid phase, and performing mixing and coagulation to prepare the UCST particle.

12. A method of preparing the hydrogel fine particle according to claim 5, comprising:
a step of introducing at least one primer among a forward primer and a reverse primer of target nucleic acid or a probe of target nucleic acid into an upper critical solution temperature (UCST) material in a liquid phase and performing mixing and coagulation to prepare the UCST particle;
a pre-polymer solution preparation step of mixing the prepared UCST particle, a hydrogel monomer, and a photo initiator to prepare a pre-polymer solution, in which the solution further comprises a primer that is not comprised in the UCST particle among the forward primer and the reverse primer when only one primer among the forward primer and the reverse primer is comprised in the UCST particle; and
a step of ejecting the pre-polymer solution in a droplet form and curing the solution to prepare the hydrogel fine particle.

13. The preparation method according to claim 12, further comprising:
a washing step after the hydrogel fine particle is prepared.

14. A nucleic acid amplification method comprising:
a step of injecting at least one UCST particle according to claim 1 into a reaction chamber;
a step of injecting a solution comprising at least one target nucleic acid into the reaction chamber; and
a step of subjecting the target nucleic acid to a polymerase chain reaction (PCR) to amplify the target nucleic acid.

15. The nucleic acid amplification method according to claim 14,
wherein the polymerase chain reaction (PCR) is a reverse transcription PCR (RT-PCR).

16. The nucleic acid amplification method according to claim 14,
wherein the step of amplifying target nucleic acid comprises melting the UCST particle in a denaturation step of PCR and releasing the primer or the probe comprised inside thereof into the chamber.

17. The nucleic acid amplification method according to claim 16,
wherein the at least one UCST particle each comprise a primer or a probe for different target nucleic acids.

18. A nucleic acid amplification method comprising:
a step of injecting the hydrogel fine particle according to claim 5 into a reaction chamber;
a step of injecting a solution comprising at least one target nucleic acid into the reaction chamber; and
a step of subjecting the target nucleic acid to a polymerase chain reaction (PCR) to amplify the target nucleic acid.

19. The nucleic acid amplification method according to claim 18,
wherein the step of amplifying target nucleic acid comprises melting the UCST particle in a denaturation step of PCR and releasing a primer or a probe comprised inside thereof into pores of the hydrogel fine particle.

20. The nucleic acid amplification method according to claim 18, further comprising:
a step of analyzing at least one nucleic acid polymerized in the hydrogel fine particle.
